(19) United States
(12) Patent Application Publication
(10) Pub. No.: US 2026/0334389 A1
(43) Pub. Date: Oct. 29, 2026

(54) METHOD FOR PRODUCING SOLID CARRIER HAVING NITRATE GENERATING ABILITY, PLANT CULTIVATION METHOD USING SOLID CARRIER, AND SOIL COMPOSITION

(71) Applicant: TOWING CO., LTD., Nagoya-shi, Aichi (JP)

(72) Inventors: Makoto SHINOHARA, Tsu-shi, Mie (JP); Ryoya NISHIDA, Nagoya-shi, Aichi (JP); Kohei NISHIDA, Nagoya-shi, Aichi (JP); Shunsuke KIMURA, Nagoya-shi, Aichi (JP); Akinori ANDO, Kyoto-shi, Kyoto (JP)

(73) Assignee: TOWING CO., LTD., Nagoya-shi, Aichi (JP)

(21) Appl. No.: 19/117,552

(22) PCT Filed: Oct. 3, 2023
(86) PCT No.: PCT/JP2023/035990

§ 371 Date: Apr. 1, 2025

(30) Foreign Application Priority Data
Oct. 3, 2022 (JP) 2022-159741

(51) Int. Cl. A01G 24/12; A01G 24/22; A01G 24/30; C12N 11/093; C12N 11/14

(52) U.S. Cl. CPC A01G 24/12; A01G 24/22; A01G 24/30; C12N 11/093; C12N 11/14

(57) ABSTRACT

Provided is a soil formation technique that immobilizes microbial populations to the entire solid carrier, and it does not require a step of washing nitrate nitrogen generated in a process of soil formation from a solid carrier. Provided is a method for producing a solid carrier having nitrate generating ability, the method comprises allowing all of the following (B) to (E) to coexist in the following solid carrier (A), in which a solid carrier to which the following microbial population (B) is immobilized is produced. The solid carrier can be used as a culture medium in any plant cultivation method. Additionally, provided is a soil composition for producing the solid carrier. (A) A solid carrier having air permeability. (B) A microbial population that generates nitrate nitrogen by mineralizing organic matter. (C) Organic matter. (D) Water. (E) A plant body that absorbs nitrate nitrogen.

### CROSS-REFERENCE TO RELATED APPLICATIONS

This application is a 35 U.S.C. § 371 national phase of PCT International Application No. PCT/JP2023/035990, filed on Oct. 3, 2023, which claims the benefit of priority under 35 U.S.C. § 119 to Japanese Patent Application No. 2022-159741, filed on Oct. 3, 2022, the contents of which are incorporated herein by reference in its entirety.

### REFERENCE TO SEQUENCE LISTING

The present disclosure incorporates by reference in its entirety the material in the accompanying XML text file “F5004-PCT Sequence Listing.xml”, created Mar. 25, 2025, having a file size of 6.31 KB.

### TECHNICAL FIELD

The present disclosure relates to a technique for forming a non-soil medium into soil, and specifically, relates to a method for producing a solid carrier having nitrate generating ability, a plant cultivation method using the solid carrier, and a soil composition.

### BACKGROUND

In natural soil, due to an action of a microbial population that decomposes organic matter to generate nitrate nitrogen, when organic matter such as compost is directly added, the organic matter is decomposed into nitrate nitrogen that can be used by many plants by a two-stage process of ammonia formation that converts organic nitrogen into ammonia nitrogen and nitrate formation (nitrification) that converts ammonia nitrogen into nitrate nitrogen.

On the other hand, in a solid carrier that is not formed into soil, nitrifying bacteria responsible for nitrate formation are not sufficiently immobilized, and thus, nitrifying bacteria are killed or roots of plants are damaged when a high concentration of organic matter is added.

One of the inventors of the present application has developed a soil formation technique in which a microbial population that decomposes organic matter to generate nitrate nitrogen is immobilized to a solid carrier such as rockwool so that the solid carrier can decompose organic matter to generate nitrate nitrogen in the same manner as in natural soil (see, for example, Patent Literature 1: JP 5686352 B2 and Non-Patent Literature 1: Jamjan Meeboon, Ryoya Nishida, Takashi Iwai, Kazuki Fujiwara, Masao Takano & Makoto Shinohara, Development of soil-less substrates capable of degrading organic nitrogen into nitrate as in natural soils, Scientific Reports (2022) 12:785).

The techniques of Patent Literature 1 and Non Patent Literature 1 are characterized in that after the microbial population is added to the solid carrier, addition of organic matter and washing with water are repeated to prevent a denitrification reaction, thereby immobilizing microorganisms to the solid carrier (see FIG. 11).

The denitrification reaction is a phenomenon in which nitrate nitrogen is reduced to a nitrous oxide gas, a nitrogen gas, or the like by denitrifying bacteria and the nitrate nitrogen is lost, and is a reaction that is likely to be induced when organic matter serving as an energy source for denitrifying bacteria and the nitrate nitrogen coexist under anaerobic conditions. In addition, in recent years, a denitrification reaction by a nitrogen conversion reaction by anammox bacteria has also been discovered. This is a phenomenon in which ammonia nitrogen and nitrite nitrogen are converted into nitrogen gas and the like and lost under anaerobic conditions.

### SUMMARY OF INVENTION

In the techniques of Patent Literature 1 and Non Patent Literature 1, the immobilization of the microbial population is limited to a surface layer portion (upper layer portion) of the solid carrier, and thus, it has been desired to develop a technique capable of forming soil up to a deep layer portion (lower layer portion). An object of the present disclosure is to provide a soil formation technique capable of obtaining more appropriate nitrate generating ability.

That is, according to the present disclosure, there is provided a method for producing a solid carrier having nitrate generating ability, the method including a step of allowing all of the following (B) to (E) to coexist in the following solid carrier (A), in which a solid carrier to which the following (B) microbial population is immobilized is produced,
(A) a solid carrier having air permeability,
(B) a microbial population that generates nitrate nitrogen by mineralizing organic matter,
(C) organic matter,
(D) water, and
(E) a plant body that absorbs nitrate nitrogen.

In the method for producing a solid carrier of the present disclosure, the plant body (E) can be allowed to coexist in the solid carrier (A) by sowing or planting a seed of the plant body (E) or a vegetatively propagatable tissue of the plant body (E) in the solid carrier (A). More specifically, in the method, after a step of adding and/or mixing the microbial population (B) to an upper layer portion of the solid carrier (A) is performed, a step of sowing or planting a seed of the plant body (E) or a vegetatively propagatable tissue of the plant body (E) in the solid carrier (A) may be performed. In addition, in the method, after a step of attaching the microbial population (B) to at least a part of a surface of the seed of the plant body (E) or a vegetatively propagatable tissue of the plant body (E) is performed, a step of sowing or planting the seed of the plant body (E) or the vegetatively propagatable tissue of the plant body (E) in the solid carrier (A) may be performed.

Alternatively, in the method for producing a solid carrier of the present disclosure, the plant body (E) can be allowed to coexist in the solid carrier (A) by planting the plant body (E) after germination in the solid carrier (A). More specifically, the method may include a step of growing the plant body (E) in soil containing the microbial population (B); and a step of planting the plant body (E) after germination obtained in the previous step in the solid carrier (A). In addition, in the method, after the step of adding and/or mixing the microbial population (B) to the upper layer portion of the solid carrier (A) is performed, the step of planting the plant body (E) after germination in the solid carrier (A) may be performed.

In this case, the solid carrier (A) may be one or more selected from the group consisting of biochar, ceramics, rockwool, vermiculite, perlite, zeolite, sand, Kanuma soil, glass, urethane, nylon, a melamine resin, a wood chip, straw, sphagnum moss, cotton, paper, polyacrylamide gel, agar, pumice, a soil material, a soil material sintered body, and field soil.

In addition, the organic matter (C) may be one or more selected from the group consisting of plant-derived, animal-derived, microorganism-derived, food-derived, and waste-derived organic matter.

In addition, the plant body (E) may be one or more plant bodies selected from Asteraceae, Brassicaceae, Solanaceae, Amaranthaceae, Amaryllidaceae, Rosaceae, Fabaceae, Cucurbitaceae, Poaceae, Zingiberaceae, Liliaceae, Apiaceae, Chenopodiaceae, and Convolvulaceae.

In addition, the solid carrier (A) can be used in a state of being filled in a container.

In addition, a cultivation method of the present disclosure is a plant cultivation method including producing a solid carrier having nitrate generating ability by the above method, and cultivating a plant using the obtained solid carrier.

Furthermore, a soil composition of the present disclosure is a soil composition for producing a solid carrier having nitrate generating ability, in which all of the (B) to (E) are allowed to coexist in the solid carrier (A).

In the present specification, the “soil composition” refers to a composition containing, as a main component, the solid carrier (A) in which soil formation is in progress, and an intermediate generated in the process of forming the solid carrier into soil.

In the soil composition, the solid carrier (A) may have a nitrate recovery rate of 1% or more as measured by the following method.

### Nitrate Recovery Rate Measuring Method:

(1) The organic matter (C) is added to the solid carrier in an amount of nitrogen of 0.05 g or more per 1 L of the solid carrier and then allowed to stand at 24 to 26° C. for 1 hour or longer.
(2) The solid carrier is washed by adding 50 mL or more of water per 1 L of the solid carrier and allowing the water to flow out from the solid carrier.
(3) An outflow liquid flowed out from the solid carrier is recovered, and a content of nitrate nitrogen in the outflow liquid is measured.
(4) A ratio of the content of nitrate nitrogen in the outflow liquid measured in the step (3) to the amount of nitrogen in the organic matter added in the step (1) is determined as a nitrate recovery rate.

In the soil composition, the solid carrier (A), the organic matter (C), and the plant body (E) may be those described above.

In the soil composition, the solid carrier (A) may be in a state of being filled in a container.

### BRIEF DESCRIPTION OF THE DRAWINGS

FIG. 1 is a flowchart showing an embodiment of a method for producing a solid carrier.

FIG. 2 is a flowchart showing an embodiment of a method for producing a solid carrier.

FIG. 3 is a schematic view illustrating an embodiment of a method for producing a solid carrier.

FIG. 4 is a schematic view illustrating an embodiment of a method for producing a solid carrier.

FIG. 5 is a graph showing a nitrate nitrogen recovery rate (%) in Example 1.

FIG. 6A is a photographic image showing a growth situation of plants in a control in Example 2.

FIG. 6B is a photographic image showing a growth situation of plants in a test section in Example 2.

FIG. 7A is a photographic image showing an appearance of a plant before harvest in Example 3.

FIG. 7B is a graph showing a measured value of an above-ground portion yield (g) in Example 3. The value indicates a mean±standard error.

FIG. 7C is a graph showing a measured value of a nitrate nitrogen recovery rate (%) in Example 3. The value indicates a mean±standard error.

FIG. 8A is a photographic image showing an appearance of a plant before harvest in Example 4.

FIG. 8B is a graph showing a measured value of an above-ground portion yield (g) in Example 4. The value indicates a mean±standard error.

FIG. 8C is a graph showing a measured value of a nitrate nitrogen recovery rate (%) in Example 4. The value indicates a mean±standard error.

FIG. 9A is a photographic image showing an appearance before harvest of a red leaf lettuce section in Example 5.

FIG. 9B is a photographic image showing an appearance of a non-plant section (control) in Example 5.

FIG. 9C is a graph showing measured values of nitrate nitrogen recovery rates (%) before and after a disturbance treatment in Example 5. “A” represents the value before the disturbance, “B” represents the value after the disturbance, and the values indicate a mean±standard error.

FIG. 10A is a photographic image showing an appearance of a plant before harvest in Example 7.

FIG. 10B is a photographic image showing an appearance of a measurement column in Example 7.

FIG. 11 is a schematic view illustrating a soil formation technique in the related art.

FIG. 12A is a schematic view illustrating a configuration of a hairy vetch section in Example 8. The solid carrier filled in a pipe is divided into eight layers L1 to L8 in order from the top.

FIG. 12B is a schematic view showing a configuration of a control in Example 8. The solid carrier filled in a pipe is divided into eight layers L1 to L8 in order from the top.

FIG. 13A is a graph showing a relative value (%) of an ammonia-oxidizing bacteria group (amoA) in each of the layers of L1 to L8 based on a total amount of bacteria of L1 in Example 8. A black bar graph indicates a control, a white bar graph indicates a hairy vetch section, and a value indicates a mean±standard error.

FIG. 13B is a graph showing a relative value (%) of a nitrite-oxidizing bacteria group (norB) in each of the layers of L1 to L8 based on a total amount of bacteria of L1 in Example 8. A black bar graph indicates a control, a white bar graph indicates a hairy vetch section, and a value indicates a mean±standard error.

FIG. 14 is a photographic image showing a growth situation of a plant in Comparative Examples.

### DETAILED DESCRIPTION

### [Method for Producing Solid Carrier Having Nitrate Generating Ability]

A first embodiment relates to a method for producing a solid carrier having nitrate generating ability.

In the present embodiment, a method for producing a solid carrier having nitrate generating ability includes a step of allowing all of the following (B) to (E) to coexist in the following solid carrier (A), in which a solid carrier to which the following microbial population (B) is immobilized is produced.
(A) A solid carrier having air permeability.
(B) A microbial population that generates nitrate nitrogen by mineralizing organic matter.
(C) Organic matter.
(D) Water.
(E) A plant body that absorbs nitrate nitrogen.

In the present embodiment, as the “solid carrier having air permeability”, any solid carrier can be used as long as it is capable of carrying microorganisms, organic components, and moisture, has air permeability, and is porous. In particular, a material having a large surface area for immobilizing (fixing) microorganisms with respect to a volume is preferable.

Here, “having air permeability” is a state in which a gas phase in which nitrate formation is likely to proceed and a denitrification reaction is unlikely to occur is secured as aerobic conditions are maintained in an environment of voids between solid carriers and/or in solid carriers when the solid carriers are filled in a container or piled up.

Note that, hereinafter, the entire environment between the solid carriers and in the solid carriers may be simply referred to as “in the solid carrier” or “the entire solid carrier”.

Specific examples of the solid carrier include a rock mineral-based material such as rockwool, vermiculite, perlite, zeolite, sand, Kanuma soil, glass (specifically, glass beads), ceramics, pumice, a soil material, a soil material sintered body, or field soil, a synthetic resin-based material such as urethane, nylon, a melamine resin, or polyacrylamide gel, and an organism-derived material such as a wood chip (specifically, Japanese cedar chip), straw, sphagnum moss, charcoal (specifically, biochar or activated carbon), cotton, paper (specifically, filter paper), or agar.

In the present embodiment, among these materials, it is desirable to use one or more selected from the group consisting of biochar, ceramics, rockwool, vermiculite, perlite, zeolite, sand, Kanuma soil, glass, urethane, nylon, a melamine resin, a wood chip, straw, sphagnum moss, cotton, paper, polyacrylamide gel, agar, pumice, a soil material, a soil material sintered body, and field soil, and biochar is particularly desirable.

Note that, here, the “biochar” is obtained by heating a biological resource (for example, agricultural waste such as chaff, thinning, bamboo, or the like) under a low oxygen condition, and is produced as a by-product of biofuel. Specific examples thereof include rice husk charcoal, charcoal, and bamboo charcoal.

In the present embodiment, the solid carrier can be used in a state of being filled in a container. As the container for filling the solid carrier, any container can be used as long as the container can be filled with the solid carrier. Specific examples thereof include a pot for agriculture, a planter, or the like; a bag or the like formed of plastic, cloth, paper, or the like; and a basket or the like in which the entire container has a mesh shape. Note that it is preferable that these containers have an opening at the bottom and have a structure in which water can flow out after adding water to the solid carrier.

The filling of the solid carrier into the container is performed in such a manner that voids (gas phases) between the solid carriers and/or in the solid carriers are secured so that the entire solid carrier is not in a water-filled state due to capillary phenomenon after water is added and caused to flow out. This is because the denitrification reaction is likely to occur when the solid carrier is in a water-filled state, and thus, the conditions become anaerobic and denitrification bacteria can easily become active. Therefore, when the container is filled with the solid carrier, usually 0.1 to 100,000 ml, and preferably 1 to 10,000 ml of the solid carrier can be filled. In addition, the solid carrier can be filled in a layer having a filling height of 0.1 to 100 cm.

In the present embodiment, the “microbial population that generates nitrate nitrogen by mineralizing organic matter (in the present specification, may be simply referred to as a “microbial population”)” includes a microbial population that performs ammonia formation and a microbial population that performs nitrate formation. When these components coexist, decomposition of organic matter into ammonia nitrogen (ammonia formation) and nitrification of ammonia nitrogen into nitrate nitrogen (nitrate formation) can be continuously performed in the same reaction system.

Examples of the microbial population that performs ammonia formation include protozoa, and ammonifying bacteria such as bacteria and filamentous fungi. Examples of the microbial population that performs nitrate formation (nitrifying bacteria) include the genus Nitrosomonas, the genus Nitorosococcus, and the genus Nitrosospira (including the genus Nitrosolobus and the genus Nitrosovibrio) which are ammonia-oxidizing bacteria (or nitrite-producing bacteria); and the genus Nitrobacter, the genus Nitrospira, and the genus Nitrococcus which are nitrite-oxidizing bacteria (or nitrate-producing bacteria).

In the present embodiment, as the “organic matter”, any organic matter such as an organic fertilizer or an organic resource such as a food residue, a plant body residue, an animal residue, a livestock waste, or an excretion can be used, and it is desirable to use high-nitrogen-containing organic matter having a C/N ratio, which is a content ratio of carbon and nitrogen, of 24 or less, and preferably 19 or less, from the viewpoint of enhancing the recovery efficiency of nitrate nitrogen.

As the organic matter, organic matter containing a large amount of protein, protein decomposition product, amino acid, and the like is desirable. Specific examples thereof include one or more kinds of plant-derived, animal-derived, microorganism-derived, food-derived, and waste-derived organic matter. More specific examples thereof include food residues such as fish broth, corn immersion liquid, oil cake, fish meal, soybean cake, yeast cake, sake cake, distilled spirit cake, rice bran, and garbage. Note that these are waste products obtained in the food production process, and are desirable in that no toxic component is contained. In addition, examples of the organic matter include excretions such as livestock feces, human feces, and insect feces, insect powder, algal powder, bacterial cells, a methane fermentation digestion liquid, and sewage sludge, and organic matter containing ammonia nitrogen can also be used. Furthermore, as the organic matter, a food itself such as soybean flour, soup stock (high amino acid content), milk, or milk powder can also be used. In addition, it is also possible to use a plant body residue which is a tissue or an organ of a plant which cannot be used as an edible portion.

Among them, one or more selected from the group consisting of fish broth, fish meal, oil cake, garbage, a corn immersion liquid, rice bran, soybean flour, a plant body residue, an animal residue, milk, milk powder, livestock feces, human feces, insect feces, insect powder, algal powder, bacterial cells, a methane fermentation digestion liquid, and sewage sludge are more desirably used as organic matter.

Note that, specifically, examples of the fish broth include skipjack broth. In addition, examples of the corn immersion liquid include corn steep liquor (CSL: corn immersion liquid which is a by-product during production of corn starch). In addition, examples of the oil cake include rapeseed oil cake and corn oil cake. In addition, examples of the plant body residue include foliage generated by a pinching treatment during cultivation management of tomatoes and the like. In addition, examples of the garbage include fish bones, cooked vegetable scraps, and meat slices. In addition, examples of the livestock feces include cow feces and chicken feces. Note that, more particularly, the skipjack broth and the corn steep liquor are desirable in that they are a liquid and thus easily permeate the solid carrier.

In the present embodiment, the “water” is not particularly limited, and examples thereof include pure water (distilled water, ion-exchanged water, reverse osmosis membrane water, or the like), well water, river water, lake water, tap water, and sea water.

In the above-described conventional technique, it is necessary to add a large amount of water to the solid carrier for washing in order to remove the nitrate nitrogen contained in the solid carrier so as to suppress the denitrification reaction. In the present embodiment, a washing step is unnecessary because allow the plant body absorbs excess nitrate nitrogen, but since water is essential for maintaining the plant body and the microbial population, it is necessary to allow an appropriate amount of water to coexist in the solid carrier.

In the present embodiment, as the “plant body that absorbs nitrate nitrogen”, any plant such as a vegetable, a fruit, a flower, a tree, a fruit tree, a foliage plant, grass, and a moss can be used. Specific examples thereof include plant bodies belonging to Asteraceae, Brassicaceae, Solanaceae, Amaranthaceae, Amaryllidaceae, Rosaceae, Fabaceae, Cucurbitaceae, Poaceae, Zingiberaceae, Liliaceae, Apiaceae, Chenopodiaceae, and Convolvulaceae.

In the present embodiment, a step of allowing all of the microbial population, the organic matter, the water, and the plant body to coexist in the solid carrier is an essential step. Note that, in the present embodiment, as long as the above-described effect is exhibited, any element other than the above-described constituent elements (for example, microorganisms other than the above-described microorganisms, media other than the above-described solid carriers, organic matter other than the above-described organic matter, inorganic fertilizers, other agricultural materials, and the like) can be allowed to coexist in the solid carrier.

In the present embodiment, examples of the means for allowing the microbial population to “coexist” in the solid carrier include adding and/or mixing a microbial source containing a microbial population to the upper layer portion of the solid carrier. Here, the “upper layer portion” of the solid carrier refers to a surface layer portion of the solid carrier, and for example, in a case of a solid carrier filled in a container, the upper layer portion can be within about 10% from the top in a height direction. Alternatively, in a case of solid carriers stacked in a field without being filled in a container, the upper layer portion may be within about 10 cm from the surface layer.

The microbial population exists in nature, and is contained in, for example, soil, tap water, water in lakes and marshes, spring water, well water, water in rivers, sea water, and the like, and thus, these natural objects can be used as a microbial source. In addition, a solid carrier formed into soil; an outflow liquid obtained by adding water to the solid carrier formed into soil and flowing out from the solid carrier; powder obtained by drying the outflow liquid; and the like can also be used as a microbial source. Note that, here, the “solid carrier formed into soil” is not limited to the solid carrier formed into soil according to the present embodiment, and may be a solid carrier to which the microbial population is immobilized by another method.

The amount of the microbial source added and/or mixed to the upper layer portion of the solid carrier is not particularly limited, and for example, in the case of natural soil or a solid carrier formed into soil, 1 to 100 g is desirably added to 1 L of the solid carrier.

In the present embodiment, as another means for allowing the microbial population to “coexist” in the solid carrier, for example, after a microbial source including the microbial population is attached to at least a part of a surface of a seed of the plant body or a vegetatively propagatable tissue of the plant body, the seed or the vegetatively propagatable tissue is sowed or planted in the solid carrier.

Here, the “vegetatively propagatable tissue of the plant body” is not particularly limited, examples thereof include a vegetative organ such as a root, a stem (including a bulb and a tuber), a leaf, and a branch of a vegetative propagating plant, and specific examples thereof include potatoes, bulbs, propagules, and runners of strawberries.

As a method for attaching the microbial population to the surface of the seed or the vegetatively propagatable tissue (hereinafter, may be referred to as a “seed or the like”), for example, a conventionally known method such as a live coating technique can be used. This step allows the microbial population and the plant body to “coexist” simultaneously in the solid carrier.

In the present embodiment, another means for allowing the microbial population to “coexist” in the solid carrier includes, for example, growing a plant body in soil containing the microbial population to attach the microbial population to the plant body, and then, planting the plant body in the solid carrier (transplanting from the soil into the solid carrier).

Here, when the plant body is planted in the solid carrier, it is desirable to plant the plant body in the solid carrier together with the soil without removing the soil in contact with the roots of the plant body. At that time, the plant body may be planted by being divided into a plurality of plants, or in the case of a vegetative propagating plant, the plant body may be planted by being divided into a plurality of tissues capable of vegetative propagation (for example, cuttings and cut shoots).

Note that the “soil” used for cultivating the plant body before planting is not limited to ordinary field soil, and may be any medium to which a microbial population is immobilized. Specifically, the solid carrier formed into soil (not limited to the solid carrier formed into soil according to the present embodiment) may be used as “soil”.

In the present embodiment, in order to allow the organic matter to “coexist” in the solid carrier, for example, the organic matter may be added to the solid carrier. Specifically, when the organic matter is a liquid, the organic matter can be directly added to the solid carrier. When the organic matter is a solid, the organic matter can be suspended or dissolved in a solvent such as water and then added to the solid carrier, or water can be added after the solid organic matter is added to the solid carrier.

Here, an addition amount of the organic matter is not particularly limited, and can be preferably 0.01 to 20 g and more preferably 0.1 to 1 g (both in terms of dry weight) with respect to 1 L of the solid carrier. Note that, specifically, the addition amount in the case of using the skipjack broth as the organic matter is preferably 0.1 to 20 g (liquid weight (0.07 to 14 g in terms of dry weight)) with respect to 1 L of the solid carrier, and the addition amount in the case of using the corn steep liquor is preferably 0.1 to 20 g (liquid weight (0.05 to 10 g in terms of dry weight)) with respect to 1 L of the solid carrier.

Note that, in a case where the addition amount of the organic matter is larger than the above addition amount, the addition amount may exceed a carrying amount of the organic matter in the solid carrier, which is not preferable. In addition, in a case where the addition amount of the organic matter is smaller than the above addition amount, a generation amount of nitrate nitrogen is reduced, which is not preferable.

An addition interval of the organic matter may be appropriately adjusted according to the type of the plant body, and can be usually 6 hours or longer, and preferably 1 to 7 days. In a case where the addition interval is shorter than the above, the decomposition of the organic matter by the microbial population cannot catch up and may exceed the carrying amount of the organic matter in the solid carrier, which is not preferable.

In the present embodiment, in order to allow water to “coexist” in the solid carrier, water may be added to the solid carrier. For example, the solid carrier can be irrigated, specific means is not particularly limited, and a conventionally known irrigation technique such as watering, spraying, or dropping can be adopted.

An addition amount of water is not particularly limited as long as the solid carrier is not in a water-filled state, and for example, it is desirable to appropriately adjust the addition amount of water within a range of 10 to 200 mL/day per 1 L of the solid carrier according to the type of the plant body or the solid carrier. In addition, the irrigation is not necessarily performed every day, and an interval of irrigation may be appropriately adjusted according to the type of the plant body or the solid carrier.

In the present embodiment, allowing the plant body to “coexist” in the solid carrier means allowing the plant body to grow in the solid carrier. Examples of the means include sowing seeds of a plant body in a solid carrier, planting a vegetatively propagatable tissue of a plant body in a solid carrier, and planting a plant body after germination in a solid carrier.

Here, the “plant body after germination” means a plant body in a state in which a bud emerges from a seed or a vegetatively propagatable tissue and is growing. Specifically, the “plant body after germination” may be a young plant body such as a plug seedling or a seedling, or may be a sufficiently grown plant body. Note that when the plant body after germination is planted in a solid carrier, the plant body may be planted by being divided into a plurality of plants, or when the plant body is a vegetative propagating plant, the plant body may be planted by being divided into a plurality of tissues capable of vegetative propagation (for example, cuttings, cut shoots, and seed potatoes).

A density of the plant body “coexisting” in the solid carrier is not particularly limited, and can be appropriately adjusted depending on the type of the plant body, for example, in a range of 1 to 10 plants per 1 L of the solid carrier.

In the present embodiment, after all of the microbial population, the organic matter, the water, and the plant body are allowed to coexist in the solid carrier as described above, the organic matter and the water are added to the solid carrier to grow the plant body. A specific aspect related to the growth (cultivation) of the plant body can be appropriately determined by those skilled in the art according to the type of the plant body or environment based on common technical knowledge.

By adding organic matter and water to the solid carrier to which the microbial population is added, the microbial population is immobilized to the solid carrier. As a result, since the microbial population can efficiently decompose the organic matter into nitrate nitrogen, the growth of the plant body is promoted, and the roots of the plant body extend to a lower layer portion of the solid carrier. The microbial population expands the habitat to the lower layer portion as the roots of the plant body extend.

In this manner, in the present embodiment, a microbial population that generates nitrate nitrogen by mineralizing organic matter is immobilized to the entire solid carrier, such that a solid carrier having nitrate generating ability can be produced.

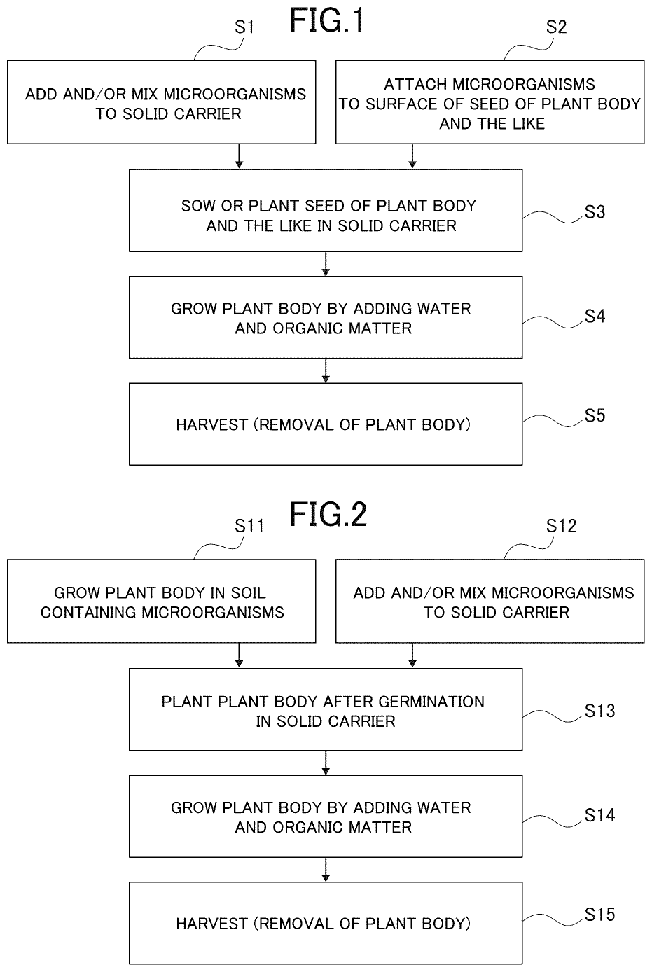

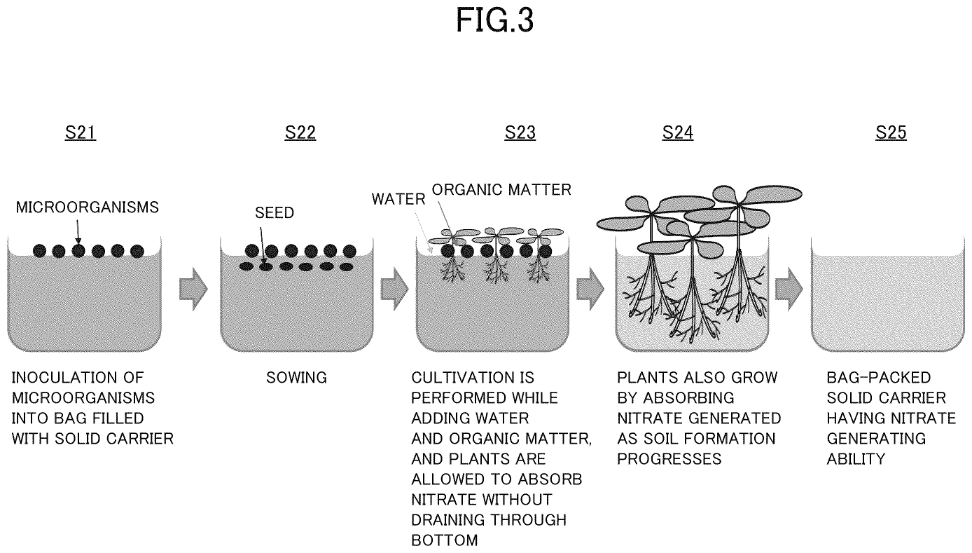

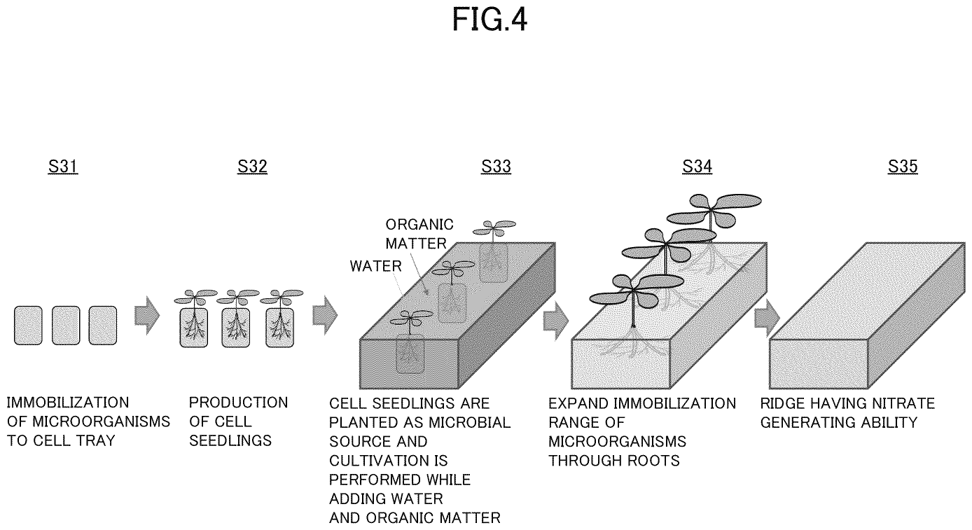

An aspect of specific steps of the present embodiment will be described in detail as follows with reference to FIGS. 1 to 4. FIGS. 1 and 2 are flowcharts showing an embodiment of the method for producing a solid carrier. FIGS. 3 and 4 are schematic views showing an embodiment of the method for producing a solid carrier.

In an embodiment illustrated in FIG. 1, first, in step S1, an operator of the method for producing a solid carrier adds and/or mixes a microbial population to an upper layer portion of the solid carrier. Here, the microbial population is added as a microbial source such as soil described above. In addition, the solid carrier that is sufficiently hydrated can be used by being filled in an appropriate container.

Next, in step S3, seeds of the plant body are sowed or a vegetatively propagatable tissue (for example, a bulb) of the plant body is planted. Note that, after the present step, a step of adding and mixing the microbial population described above can also be performed in the solid carrier to which the microbial source is added.

Then, in step S4, water and organic matter (fertilizer) are added to the solid carrier to grow the plant body. The water and the organic matter may be added in a mixed state or may be added separately. By the addition of the organic matter, the microbial population is also grown and immobilized to the upper layer portion, and the organic matter can be efficiently decomposed into nitrate nitrogen. Since the generated nitrate nitrogen is absorbed from the roots of plants, the denitrification reaction hardly occurs without washing with a large amount of water. As the plant body grows, the roots extend, and the microbial population that inhabits the upper layer portion of the solid carrier expands in the solid carrier as a rhizosphere develops.

Finally, in step S5, the sufficiently grown plant body is harvested (removal of the plant body). When the roots of the plant body extend in the solid carrier, it can be seen that the microbial population is immobilized to the entire solid carrier (soil formation is sufficiently performed).

In another embodiment illustrated in FIG. 1, first, in step S2, a microbial population is attached to at least a part of a surface of a seed of a plant body or a vegetatively propagatable tissue (for example, a bulb) of the plant body. Here, the microbial population can be attached to the surface of the seed or the like as the above-described microbial source (for example, an outflow liquid from the solid carrier formed into soil, or the like).

Next, in step S3, the seeds to which the microbial source is attached are sowed in the solid carrier, or the vegetatively propagatable tissue of the plant body to which the microbial source is attached is planted in the solid carrier. Here, the solid carrier that is sufficiently hydrated can be used by being filled in an appropriate container.

Thereafter, similarly to the above, water and organic matter (fertilizer) are added to the solid carrier to grow the plant body (step S4), and the sufficiently grown plant body is harvested (step S5).

In an embodiment illustrated in FIG. 2, first, in step S11, a plant body is grown in soil containing a microbial population. Here, as described above, the “soil” containing a microbial population does not refer to general soil, and any medium may be used as long as the medium contains a microbial population immobilized thereto, and a solid carrier formed into soil may be used as the “soil”.

Next, in step S13, the plant body after germination (for example, plug seedling or the like) grown in step S11 is planted in a solid carrier. Here, the solid carrier that is sufficiently hydrated can be used by being filled in an appropriate container. Note that, at the time of planting, it is desirable to transplant the plant body together with the soil in contact with the roots of the plant body. In addition, the plant body may be planted by being divided into a plurality of plants.

Thereafter, similarly to the above, water and organic matter (fertilizer) are added to the solid carrier to grow the plant body (step S14), and the sufficiently grown plant body is harvested (removal of the plant body) (step S15).

In another embodiment illustrated in FIG. 2, first, in step S12, the microbial population is added and/or mixed to the upper layer portion of the solid carrier. Here, the microbial population can be added as a microbial source such as soil described above. In addition, the solid carrier that is sufficiently hydrated can be used by being filled in an appropriate container.

Next, in step S13, the plant body after germination (for example, plug seedling or the like) separately grown is planted in a solid carrier. Note that, after the present step, a step of adding and mixing the microbial population described above can also be performed.

Thereafter, similarly to the above, water and organic matter (fertilizer) are added to the solid carrier to grow the plant body (step S14), and the sufficiently grown plant body is harvested (step S15).

In an embodiment illustrated in FIG. 3, first, a bag is filled with the solid carrier, and the surface of the solid carrier is inoculated with a small amount of a microbial source such as culture soil (step S21). Next, the seeds of the plant body are sowed in the solid carrier (step S22). Then, the plant body is cultivated while adding water and organic matter (step S23). Since the microbial population decomposes the organic matter to produce nitrate nitrogen and causes the nitrate nitrogen to be absorbed by the roots of the plant body, it is not necessary to wash the nitrate nitrogen with a large amount of water.

Thereafter, the plant body continues to grow by absorbing nitrate nitrogen, and the roots also extend to the lower layer portion of the solid carrier (step S24). The grown microbial population expands the habitat in the entire solid carrier along with the development of the roots of the plant. Finally, when the sufficiently grown plant body is removed, a bag-packed solid carrier having nitrate generating ability is obtained (step S25).

In an embodiment illustrated in FIG. 4, first, in step S31, a cell tray is filled with a microbial source such as culture soil (immobilization of microorganisms), and in step S32, the seeds of the plant body are sowed thereto and raised to produce cell seedlings. Next, in step S33, the cell seedlings are planted on ridges formed of the solid carrier, and the plant bodies are cultivated while adding water and organic matter. The cell seedling itself serves as a microbial source, and the microbial population grows in the solid carrier. Since the microbial population decomposes the organic matter to produce nitrate nitrogen and causes the nitrate nitrogen to be absorbed by the roots of the plant body, it is not necessary to wash the nitrate nitrogen with a large amount of water.

Thereafter, in step S34, the plant body continues to grow by absorbing nitrate nitrogen, and the roots also extend widely in the ridges of the solid carrier. The grown microbial population expands an immobilization range throughout the ridges as the roots of the plant develop. Finally, in step S35, when the sufficiently grown plant body is removed, the ridges of the solid carrier having nitrate generating ability are obtained.

In the present embodiment, whether or not the microbial population is immobilized to the solid carrier and the solid carrier is formed into soil can be confirmed by evaluating the nitrate generating ability of the solid carrier. The nitrate generating ability can be evaluated by measuring a nitrate recovery rate (%) by the following method.

### Nitrate Recovery Rate Measuring Method:

(1) The organic matter is added to the solid carrier in an amount of nitrogen of 0.05 g or more per 1 L of the solid carrier and then allowed to stand at 24 to 26° C. for 1 hour or longer and preferably 6 hours or longer. During the standing period, the organic matter is decomposed by the microbial population to generate nitrate nitrogen.
(2) The solid carrier is washed by adding 50 mL or more of water per 1 L of the solid carrier and allowing the water to flow out from the solid carrier.
(3) An outflow liquid flowed out from the solid carrier is recovered, and a content of nitrate nitrogen (nitrate ion) in the outflow liquid is measured.
(4) A ratio of the content of nitrate nitrogen in the outflow liquid measured in the step (3) to the amount of nitrogen in the organic matter added in the step (1) is determined as a nitrate recovery rate (%).

When the nitrate recovery rate obtained by the above method is usually 30% or more, preferably 33% or more, more preferably 35% or more, still more preferably 37% or more, and particularly preferably 40% or more, the nitrate recovery rate can be evaluated that the solid carrier can be formed into soil normally. On the other hand, when the nitrate recovery rate is 1% or more, preferably 1% or more and less than 30%, and more preferably 1% or more and less than 20%, there is a possibility that soil formation is in progress and microflora is unstable, or a denitrification reaction occurs and nitrate nitrogen is lost.

In addition, the soil formation can also be confirmed by confirming the extension of the roots of the plant body in the solid carrier. This is because nitrate nitrogen that can be used by the plant body is not generated in a region where the microbial population (particularly nitrifying bacteria) is not immobilized, and thus, the roots are exposed to the organic matter and damaged. Therefore, when the roots extend over the entire solid carrier, it can be confirmed that the microbial population is immobilized to the entire solid carrier.

Note that, in the process of soil formation, microflora becomes unstable, and the measured value of the nitrate recovery rate may be temporarily 30% or more. Therefore, it is desirable that the measurement of the nitrate recovery rate described above is performed by collecting a plurality of samples from different regions (preferably, three or more locations from the region below a middle layer of the solid carrier) in the solid carrier, and the soil formation is evaluated by a mean thereof. Alternatively, it is also possible to comprehensively evaluate soil formation by combining the plurality of evaluation methods described above.

In the solid carrier having nitrate generating ability produced according to the present embodiment, as described below in Examples, the microbial population is immobilized to the entire solid carrier, and the nitrate generating ability can be maintained at a high level even when the disturbance treatment is performed. Therefore, even when the solid carrier is divided and enclosed in an appropriate container, there is little variation in quality, and the solid carrier can be distributed on the market.

### [Method for Cultivating Plant Using Solid Carrier]

The solid carrier having nitrate generating ability can decompose organic matter into nitrate nitrogen in a short time even when the organic matter is directly added to the solid carrier, and thus can be used as a plant cultivation medium that can be applied to any plant cultivation method.

That is, the present embodiment relates to a plant cultivation method including producing a solid carrier having nitrate generating ability by the above method, and cultivating a plant using the obtained solid carrier.

Specifically, the method for cultivating a plant using a solid carrier having nitrate generating ability described above can be performed by sowing seeds of a plant, planting a vegetatively propagatable tissue of the plant (for example, a bulb or the like), or planting a plant body after germination (for example, plug seedling or the like) to the solid carrier, and directly adding water and an organic fertilizer to the solid carrier.

Here, as the organic fertilizer, the above-described organic matter can be used. The organic fertilizer can be used either in a liquid form or in a powder form, and a liquid organic fertilizer, particularly fish broth or corn steep liquor, or an aqueous suspension of a powdered organic fertilizer can be used preferably. An addition amount of the organic fertilizer is as described in the method for producing a solid carrier.

The cultivation method of the present embodiment can be applied to all plants such as vegetables, fruits, flowers, trees, fruit trees, and foliage plants. Specific examples thereof include plant bodies belonging to Asteraceae, Brassicaceae, Solanaceae, Amaranthaceae, Amaryllidaceae, Rosaceae, Fabaceae, Cucurbitaceae, Poaceae, Zingiberaceae, Liliaceae, Apiaceae, Chenopodiaceae, and Convolvulaceae.

The solid carrier having nitrate generating ability can be used as a carrier of a catalyst column for generating nitrate nitrogen (nitrate ion or nitrate) from organic matter in addition to the above-described plant cultivation method. That is, nitrate nitrogen can be obtained by filling a column with a solid carrier, adding liquid organic matter or an aqueous suspension of powder organic matter to the column, and recovering an outflow liquid that has passed through the solid carrier. The obtained liquid containing nitrate nitrogen can be used as an inorganic fertilizer. Here, as the organic matter added to the column, the above-described organic matter can be used.

### [Soil Composition (Intermediate) for Producing Solid Carrier]

A further embodiment relates to a soil composition for producing a solid carrier having nitrate generating ability, in which all of the (B) to (E) are allowed to coexist in the solid carrier (A). Here, as described above, the “soil composition” refers to a composition containing, as a main component, the solid carrier in which soil formation is in progress, and an intermediate generated in the process of forming the solid carrier into soil. In addition, the respective elements (A) to (E) are as described above.

The solid carrier of the soil composition in the present embodiment is only required to have a certain level of nitrate generating ability, and sufficient nitrate generating ability may be obtained through the above-described method for producing a solid carrier. As a criterion for evaluating the nitrate generating ability, a nitrate recovery rate (%) measured by the above-described nitrate recovery rate measurement method can be used. That is, when the nitrate recovery rate measured for the solid carrier of the soil composition is 1% or more, preferably 1% or more and less than 30%, and more preferably 1% or more and less than 20%, it can be determined that “soil formation is in progress”.

In addition, as described above, the soil formation can also be confirmed by confirming the state of the roots of the plant body in the solid carrier. This is because nitrate nitrogen that can be used by the plant body is not generated in a region where the microbial population (particularly nitrifying bacteria) is not immobilized, and thus, the roots are exposed to the organic matter and damaged. Therefore, when root development in the solid carrier is insufficient, it can be determined that “soil formation is in progress”.

Note that, as described above, in the process of soil formation, microflora becomes unstable, and the measured value of the nitrate recovery rate may be temporarily high. Therefore, it is desirable that the measurement of the nitrate recovery rate described above is performed by collecting a plurality of samples from different regions (preferably, three or more locations from the region below a middle layer of the solid carrier) in the solid carrier, and the soil formation is evaluated by a mean thereof. Alternatively, it is also possible to comprehensively evaluate soil formation by combining the plurality of evaluation methods described above.

In the soil composition in the present embodiment, the soil formation is in progress and its microflora is unstable, but at least the solid carrier in the upper layer portion is considered to be formed into soil. By using such a soil composition, a solid carrier with excellent nitrate generating ability can be produced. In addition, the soil composition may be filled in a suitable container. The soil composition filled in the container is easy to transport and can be distributed on the market. Therefore, since the soil composition can be preferably used for the above-described plant cultivation medium or the like, it is industrially applicable.

The present disclosure is specifically described as follows with reference to Examples and Comparative Examples. Note that, in the following description, “%” means “mass %” unless otherwise specified.

### Example 1

We conducted a soil formation test using coriander (Apiaceae), sunflower (Asteraceae), and lettuce (Asteraceae).

### (Test Method)

(1) Into a vinyl pot having a diameter of 9 cm, 300 mL of sufficiently hydrated rice husk charcoal as a solid carrier was added, and 3 g of bark compost (trade name: SANYO BARK, Sanyo Chip Industry Co., Ltd.) as a microbial source was spread thereon. Furthermore, from above, a liquid obtained by suspending 0.3 g (liquid mass, equivalent to 18 mg N, C/N ratio 2.9, and the same applies below) of skipjack broth, which is a by-product of a dried bonito plant as organic matter in 10 ml of water was added.
(2) Five seeds of coriander, sunflower, or lettuce were sowed in the pots (4 pots for each group).
(3) For each pot, 20 ml of water was charged daily without drainage from the bottom of the pot to cultivate plants.
(4) After developing the true leaf, a liquid obtained by suspending 0.3 g of the skipjack broth in 10 ml of water was added to each pot once a week after the watering described in the (3) above, and cultivation was performed for 6 weeks.
(5) After 6 weeks of cultivation, the plant body was removed together with the roots and harvested, and 600 ml of water was added to each pot to wash the solid carrier.
(6) A nitrate nitrogen recovery rate was measured by the following method. The liquid obtained by suspending 0.3 g of the skipjack broth in 10 ml of water was added again to each pot and then allowed to stand at 25° C. for 1 day. Thereafter, 300 mL of water was added to each pot, and the amount of nitrate nitrogen in an outflow liquid flowing out from the bottom of the pot was measured. The amount of nitrate nitrogen (nitrate ion concentration) was measured by RQ Flex (Merck KGaA) using Reflect Quant (registered trademark) nitrate test (Merck KGaA) (the same applies below).

### Results

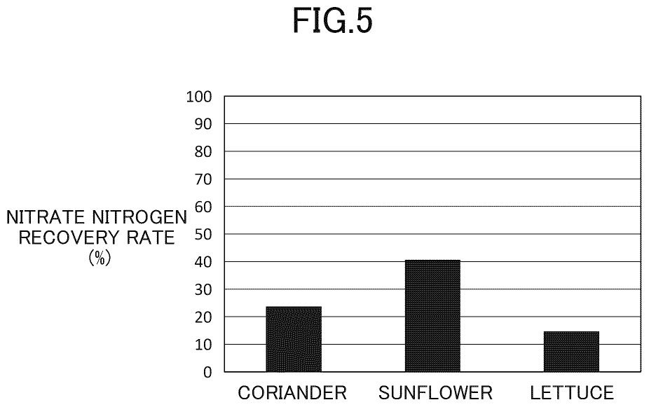

FIG. 5 illustrates a nitrate nitrogen recovery rate (%) obtained by dividing a measured value of the amount of nitrate nitrogen by an N amount of the skipjack broth added in the (6) above. The sunflower, coriander, and lettuce had higher nitrate nitrogen recovery rates in this order (FIG. 5). The highest sunflower showed a nitrate nitrogen recovery rate of about 40%, indicating that a solid carrier having sufficient nitrate generating ability can be produced. On the other hand, the growth of the lettuce was good, but the nitrate nitrogen recovery rate was slightly low at 20% or less. However, there is a possibility that the recovery rate can be improved by increasing an addition amount of the organic matter.

From the above results, it was shown that by allowing the microbial population, the organic matter, the water, and the plant body to coexist in the solid carrier and cultivating the plant body, the solid carrier can be formed into soil without washing with a large amount of water several times. In addition, it was shown that the soil formation according to the present embodiment is possible using various plants.

### Example 2

A soil formation test was conducted using Japanese mustard spinach (Brassicaceae).

### (Test Method)

(1) A mesh basket was filled with 3 L of sufficiently hydrated rice husk charcoal as a solid carrier, and 30 g of culture soil (trade name: “TSUCHITARO”, Sumirin-Nosan-Kogyo Co. Ltd.) as a microbial source was spread thereon. A section treated in the same manner except that the culture soil was not spread was used as a control.
(2) To the (1) above, 30 mL of 10-fold diluted skipjack broth was sprayed as organic matter and allowed to stand at 25° C. for 2 weeks, thereby forming a surface layer portion of the rice husk charcoal into soil.
(3) 20 seeds of Japanese mustard spinach were sowed per basket after standing (3 baskets in each group).
(4) After germination, Japanese mustard spinach was cultivated for 4 weeks by irrigating daily a liquid obtained by suspending 2 g (liquid mass, equivalent to 120 mg N) of skipjack broth in 1 L of water per basket.
(5) After the end of the cultivation, a growth status of the Japanese mustard spinach and extension of roots of the Japanese mustard spinach in the solid carrier were observed.

### Results

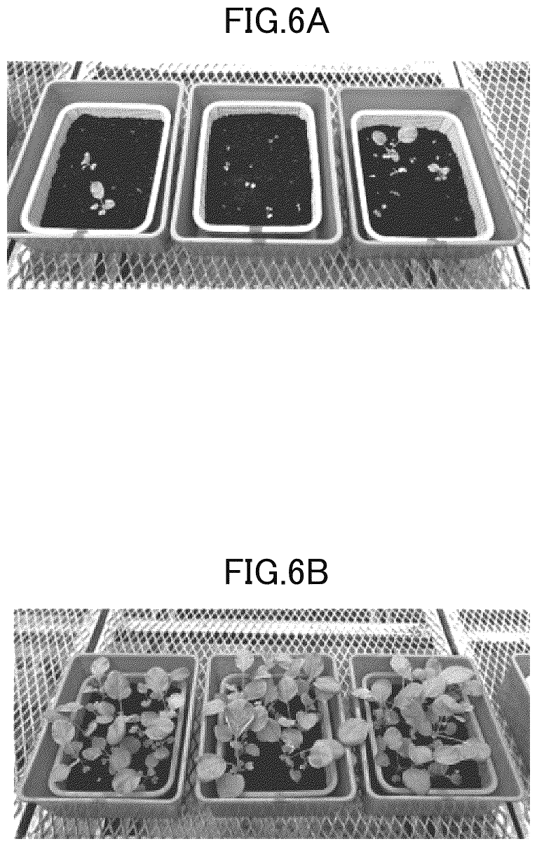

FIG. 6 is a photographic image showing a growth situation of Japanese mustard spinach at the end of cultivation. FIG. 6A illustrates a control, and FIG. 6B illustrates a test section. In the control, it was shown that, since the culture soil as a microbial source was not spread, soil formation was not performed, and the Japanese mustard spinach was not grown (FIG. 6A). On the other hand, in the test section, the Japanese mustard spinach were grown soundly, and the roots extended over the entire rice husk charcoal (FIG. 6B).

In the control, it was considered that, since a microbial population (in particular, nitrifying bacteria) that decomposes organic matter to generate nitrate nitrogen was not immobilized, nitrate nitrogen that can be used by the plant body was not generated well, and therefore, roots were damaged by exposure to the organic matter. On the other hand, in the test section, it was found that the microbial population was immobilized to the entire solid carrier because the roots extended over the entire solid carrier. From these results, it was suggested that the habitat of the microbial population was expanded over the entire solid carrier along with the extension of the roots of the plant body.

### Example 3

A soil formation test was conducted using red leaf lettuce (Asteraceae).

### (Test Method)

(1) A hole in the bottom of a vinyl pot having a diameter of 9 cm was closed, and 300 mL of sufficiently hydrated rice husk charcoal as a solid carrier was added in the pot. 10 g of culture soil (trade name: “TSUCHITARO”, Sumirin-Nosan-Kogyo Co. Ltd.) as a microbial source was spread thereon. Furthermore, from above, a liquid obtained by suspending 0.3 g (liquid mass, equivalent to 18 mg N, and the same applies below) of skipjack broth as organic matter in 10 mL of water was added.
(2) Three of the above pots were prepared, and six seeds of red leaf lettuce were sowed in each pot.
(3) A weight of each pot was measured every day, and 10 to 50 ml of water was added to each pot every day so as to replenish the weight lightened by drying, and the plants were cultivated.
(4) After true leaf development, the number of plants was reduced to three per pot. In addition, on weekdays (Monday to Friday), after the plants were watered as described in the (3) above, a liquid obtained by suspending 0.3 g of the skipjack broth in 10 ml of water was added to each pot every day, and cultivation was performed for 6 weeks.
(5) After 6 weeks of cultivation, only an above-ground portion of the red leaf lettuce was harvested, and 900 ml of water was added to each pot to wash the solid carrier.
(6) A nitrate nitrogen recovery rate was measured by the following method. The liquid obtained by suspending 0.3 g of the skipjack broth in 10 ml of water was added again to each pot and then allowed to stand at 25° C. for 1 day. Thereafter, 900 ml of water was added to each pot in a state where the hole at the bottom was opened, and the amount of nitrate nitrogen in an outflow liquid flowing out from the bottom of the pot was measured. A nitrate nitrogen recovery rate (%) was determined by dividing a measured value of the amount of nitric acid nitrogen by an N amount of the added skipjack broth (the same applies to the following Examples).

### Results

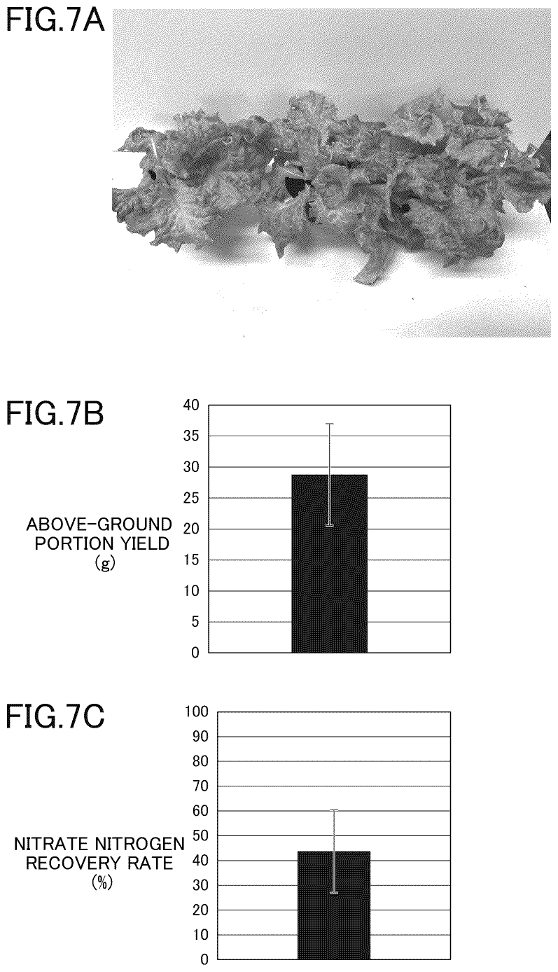

The results are illustrated in FIG. 7. FIG. 7A is a photographic image showing an appearance of a plant before harvest. FIG. 7B is a graph showing an above-ground portion yield (g), and FIG. 7C is a graph showing a nitrate nitrogen recovery rate (%), and the values indicate a mean±standard error. The growth of the red leaf lettuce was good (FIGS. 7A and 7B). In addition, the nitrate nitrogen recovery rate was 40% or more, which was significantly high (FIG. 7C). These results showed that a solid carrier having sufficient nitrate generating ability can be produced according to the present embodiment.

### Example 4

A soil formation test was conducted with a different timing for starting the addition of organic matter from that in Example 3.

### (Test Method)

(1) A hole in the bottom of a vinyl pot having a diameter of 9 cm was closed, and 300 mL of sufficiently hydrated rice husk charcoal as a solid carrier was added in the pot. 10 g of culture soil (trade name: “TSUCHITARO”, Sumirin-Nosan-Kogyo Co. Ltd.) as a microbial source was spread thereon.
(2) Three of the above pots were prepared, and six seeds of red leaf lettuce were sowed in each pot.
(3) A weight of each pot was measured every day, and 10 to 50 ml of water was added to each pot every day so as to replenish the weight lightened by drying, and the plants were cultivated.
(4) After true leaf development, the number of plants was reduced to three per pot. In addition, on weekdays (Monday to Friday), after the plants were watered as described in the (3) above, a liquid obtained by suspending 0.3 g (liquid mass, equivalent to 18 mg N, and the same applies below) of the skipjack broth in 10 mL of water was added to each pot every day, and cultivation was performed for 6 weeks.
(5) After 6 weeks of cultivation, only an above-ground portion of the red leaf lettuce was harvested, and 900 ml of water was added to each pot to wash the solid carrier.
(6) A nitrate nitrogen recovery rate was measured by the following method. The liquid obtained by suspending 0.3 g of the skipjack broth in 10 ml of water was added again to each pot and then allowed to stand at 25° C. for 1 day. Thereafter, 900 mL of water was added to each pot in a state where the hole at the bottom was opened, and the amount of nitrate nitrogen in an outflow liquid flowing out from the bottom of the pot was measured.

### Results

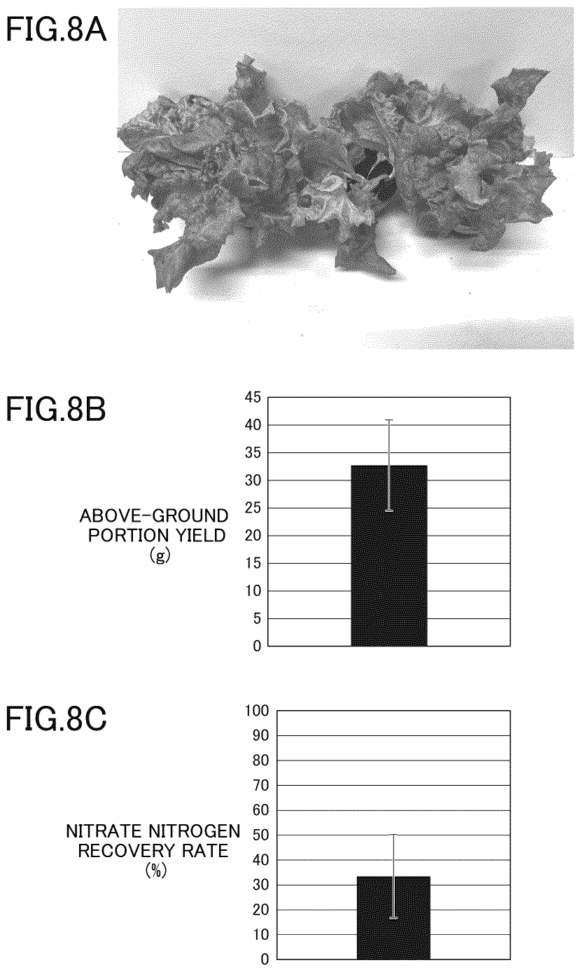

The results are illustrated in FIG. 8. FIG. 8A is a photographic image showing an appearance of a plant before harvest. FIG. 8B is a graph showing an above-ground portion yield (g), and FIG. 8C is a graph showing a nitrate nitrogen recovery rate (%), and the values indicate a mean±standard error. The growth of the red leaf lettuce was good (FIGS. 8A and 8B). In addition, the nitrate nitrogen recovery rate was 30% or more (FIG. 8C).

In Example 3, organic matter was added also before sowing, but in this example, the organic matter was not added before sowing, and the addition of the organic matter was started after development of the true leaf. Nevertheless, the nitrate nitrogen recovery rate was at a sufficient level (FIG. 8C), and it was shown that a solid carrier having sufficient nitrate generating ability can also be produced according to the present embodiment.

### Example 5

The presence or absence of a plant body and the influence of disturbance after soil formation were examined.

### (Test Method)

(1) A hole in the bottom of a vinyl pot having a diameter of 9 cm was closed, and 300 mL of sufficiently hydrated rice husk charcoal as a solid carrier was added in the pot. 10 g of culture soil (trade name: “TSUCHITARO”, Sumirin-Nosan-Kogyo Co. Ltd.) as a microbial source was spread thereon. Furthermore, from above, a liquid obtained by suspending 0.3 g (liquid mass, equivalent to 18 mg N, and the same applies below) of skipjack broth in 10 ml of water as organic matter was added.
(2) Six of the above pots were prepared, and six seeds of the red leaf lettuce were sowed on three of the pots (red leaf lettuce section). The remaining three pots were treated in the same manner except that seeds were not sowed and served as a control (non-plant section).
(3) A weight of each pot was measured every day, and 10 to 50 ml of water was added to each pot every day so as to replenish the weight lightened by drying, and the plants were cultivated.
(4) After true leaf development, the number of plants was reduced to three per pot. In addition, on weekdays (Monday to Friday), after the plants were watered as described in the (3) above, a liquid obtained by suspending 0.3 g of the skipjack broth in 10 ml of water was added to each pot every day, and cultivation was performed for 5 weeks.
(5) After 5 weeks of cultivation, only an above-ground portion of the red leaf lettuce was harvested, and 900 mL of water was added to each pot to wash the solid carrier.
(6) A nitrate nitrogen recovery rate was measured by the following method. The liquid obtained by suspending 0.3 g of the skipjack broth in 10 ml of water was added again to each pot and then allowed to stand at 25° C. for 1 day. Thereafter, 900 mL of water was added to each pot in a state where the hole at the bottom was opened, and the amount of nitrate nitrogen in an outflow liquid (outflow liquid A) flowing out from the bottom of the pot was measured.
(7) Next, all the solid carriers formed into soil by the above method were taken out from the pots, the roots of the red leaf lettuce were removed, and then, the solid carriers were returned to the pots again (disturbance treatment). For the control, similarly, the solid carriers were taken out of the pots and returned to the pots again.
(8) The operation of the (6) above was repeated for the solid carrier after disturbance, and the amount of nitrate nitrogen in the obtained outflow liquid (outflow liquid B) was measured.

### Results

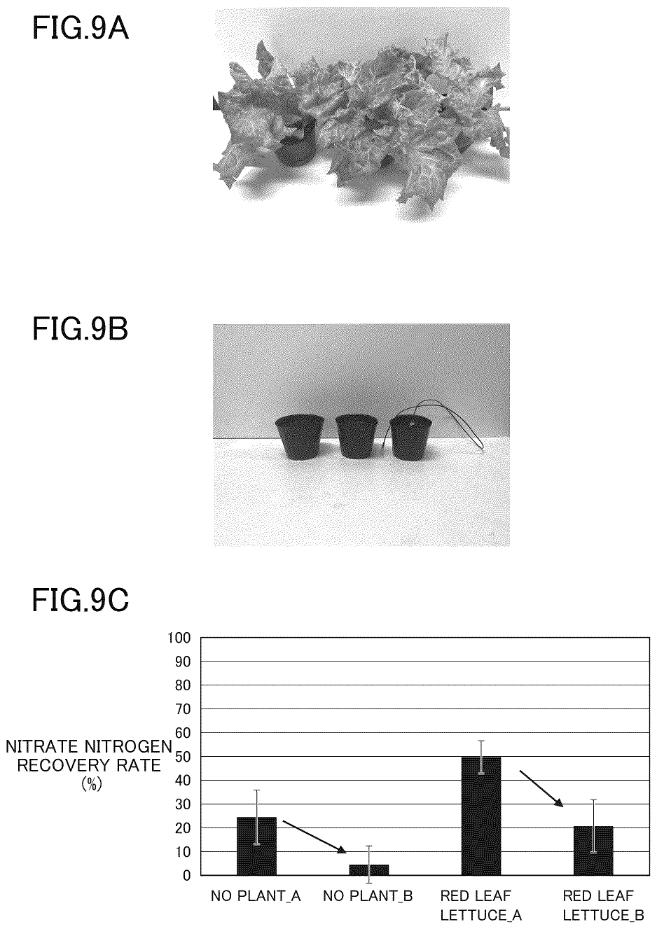

The results are illustrated in FIG. 9. FIG. 9A is a photographic image showing an appearance of a red leaf lettuce section before harvest, and FIG. 9B is a photographic image showing an appearance of a non-plant section (control). FIG. 9C is a graph showing a nitrate nitrogen recovery rate (%) before and after the disturbance treatment, where “A” indicates before the disturbance, “B” indicates after the disturbance, and the values indicate a mean±standard error. The growth of the plant in the red leaf lettuce section was good (FIG. 9A). In the red leaf lettuce section, the nitrate generating ability after the disturbance was about 40% of that before the disturbance. On the other hand, in the non-plant section (control), after disturbance, the nitrate generating ability decreased to about 16% of that before disturbance (FIG. 9C).

From the above results, it was suggested that, when soil formation was conducted by allowing a plant body to coexist, as long as the immobilization of the microbial population on the entire solid carrier was completed, the nitrate nitrogen recovery rate can be maintained at a high value without significantly falling even after the disturbance treatment is performed. On the other hand, it was shown that in the case of no plant, by spreading a microbial population and repeatedly giving organic matter and water, only the surface layer portion can be formed into soil, but the lower layer portion cannot be formed into soil, and the nitrate generating ability was significantly reduced after the disturbance (FIG. 9).

### Example 6

A soil formation test in an embodiment in which a plant body after germination is planted was conducted.

### (Test Method)

(1) A cell tray having a cell size of 3×3×6 cm was filled with culture soil (trade name: “TSUCHITARO”, Sumirin-Nosan-Kogyo Co. Ltd.) as a microbial source and sufficiently hydrated. Seeds of lettuce were sowed one by one in each cell and raised by giving only water for two weeks until true leaf development.
(2) A 15 L planter was filled with rice husk charcoal as a solid carrier, and 10 plug seedlings obtained in the (1) above were planted per planter. Thereafter, a liquid obtained by suspending 1 g (liquid mass, equivalent to 60 mg N) of the skipjack broth as organic matter in 10 ml of water was added to the plant foot every weekday to cultivate plants. Irrigation was appropriately performed.
(3) Plants grown to sufficient size were harvested. The presence or absence of nitrate ions in an outflow liquid flowing out from the bottom of the planter at the time of irrigation was detected using Reflect Quant (registered trademark) nitrate test (Merck KGaA).

### Results

The growth of the lettuce was good. Nitrate ions were detected from the outflow liquid at the time of irrigation. From these results, it was suggested that the microbial population contained in the soil of the plug seedlings was expanded and immobilized to the entire solid carrier together with the spread of the rhizosphere accompanying the growth of the lettuce. In addition, it was shown that soil formation can be conducted by planting a plant body after germination in a solid carrier that is not formed into soil, and cultivating the plant by adding organic matter and water.

### Example 7

The nitrate generating ability of the upper layer portion and the lower layer portion of the solid carrier after soil formation was confirmed.

### (Test Method)

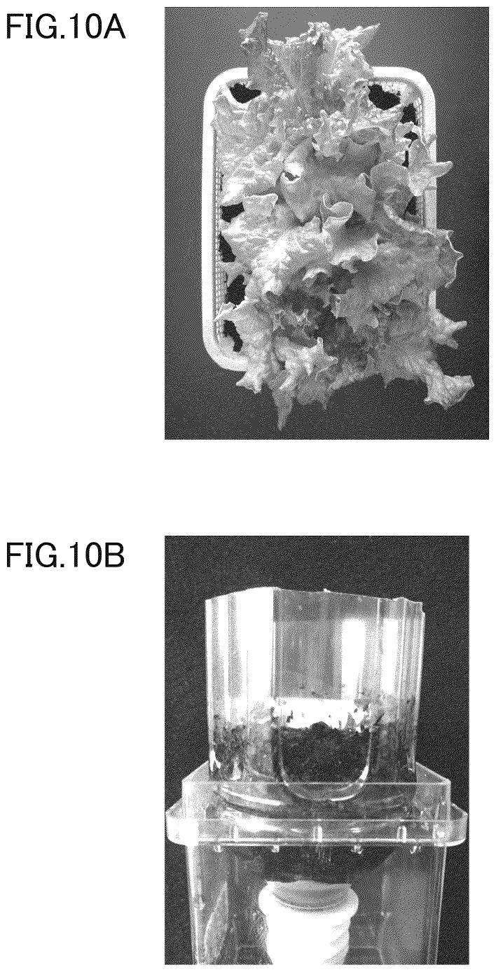

(1) A cell tray having a cell size of 3×3×6 cm was filled with culture soil (trade name: “TSUCHITARO”, Sumirin-Nosan-Kogyo Co. Ltd.) as a microbial source and sufficiently hydrated. Seeds of red leaf lettuce were sowed one by one in each cell and raised by giving only water for two weeks until true leaf development.
(2) A mesh basket was filled with 2 L of sufficiently hydrated rice husk charcoal as a solid carrier, and a tray was placed under the mesh basket. Two plug seedlings obtained in the (1) above were planted in the mesh basket. Thereafter, a liquid obtained by suspending 0.1 g (liquid mass, equivalent to 6 mg N, and the same applies below) of skipjack broth as organic matter in 100 ml of water was irrigated every day to cultivate plants. Note that, as a control, lettuce seedlings were treated in the same manner except that they were grown in a cell tray filled with vermiculite instead of culture soil and then planted.
(3) Plants grown to sufficient size were harvested. The remaining rice husk charcoal was separated into an upper layer and a lower layer, and filled into columns (FIG. 10B, prepared by cutting off an upper portion of a 2 L PET bottle), respectively. A liquid obtained by suspending 0.1 g of skipjack broth in 100 ml of water was added to each column and allowed to stand at 25° C. overnight.
(4) After standing, 100 ml of water was added to each column, and an outflow liquid was collected from a lower opening. A concentration of each of ammonium ions, nitrite ions, and nitrate ions were measured for the outflow liquid. The measurement was performed by RQ Flex (manufactured by Merck KGaA) using an ammonia test, a nitrite test, and a nitrate test of Reflect Quant (registered trademark). Note that, for comparison, the upper layer and the lower layer of the test section and the control were filled together in the column, and the measurement was performed in the same manner.

### Results

The results are illustrated in FIG. 10 and shown in Table 1. FIG. 10A is a photographic image showing an appearance of a plant before harvest, and FIG. 10B is a photographic image showing an appearance of a measurement column. The growth of the red leaf lettuce in the test section was good (FIG. 10A), whereas the control in which seedlings grown in the cell tray of vermiculite were planted did not grow soundly.

Table 1 shows the mean (unit: mg/L) of the measured values (n=3) of the ammonium ions, the nitrite ions, and the nitrate ions for the upper layer and the lower layer of the solid carrier after soil formation. In the table, “Low” refers to being below the detection limit. In the test section, generation of nitrate nitrogen was confirmed from both the columns of upper and lower layers. In addition, in the test section, no significant difference was observed in each ion concentration in any of the columns of the upper layer, the lower layer, and the upper layer+the lower layer. On the other hand, in the control, generation of nitrate nitrogen was not detected, and only ammonia nitrogen was detected at a high concentration.

TABLE 1

| | Ammonia nitrogen | Nitrite nitrogen | Nitrate nitrogen |
|---|---|---|---|
| | (unit: mg/L) | (unit: mg/L) | (unit: mg/L) |
| Upper layer + | 0.23 | 19.5 | 176 |
| lower layer | | | |
| Upper layer | 0.33 | 12.5 | 132 |
| Lower layer | 0.21 | 26.0 | 182 |
| Control | 26.9 | Low | Low |

From these results, it was shown that as the microbial population, the organic matter, the water, and the plant body were allowed to coexist in the solid carrier and the plant body was cultivated, the entire solid carrier was formed into soil evenly, and the lower layer portion alone had sufficient nitrate generating ability. In addition, it was shown that when a microbial population is not exist, soil formation is not possible even when a plant body is cultivated in a solid carrier.

### Example 8

Expansion of a nitrifying bacteria group (ammonia oxidizing bacteria and nitrite oxidizing bacteria) into a solid carrier accompanying expansion of a rhizosphere of a hairy vetch of a Fabaceae plant was evaluated by comparison with a control in which a plant was not planted.

### (Test Method)

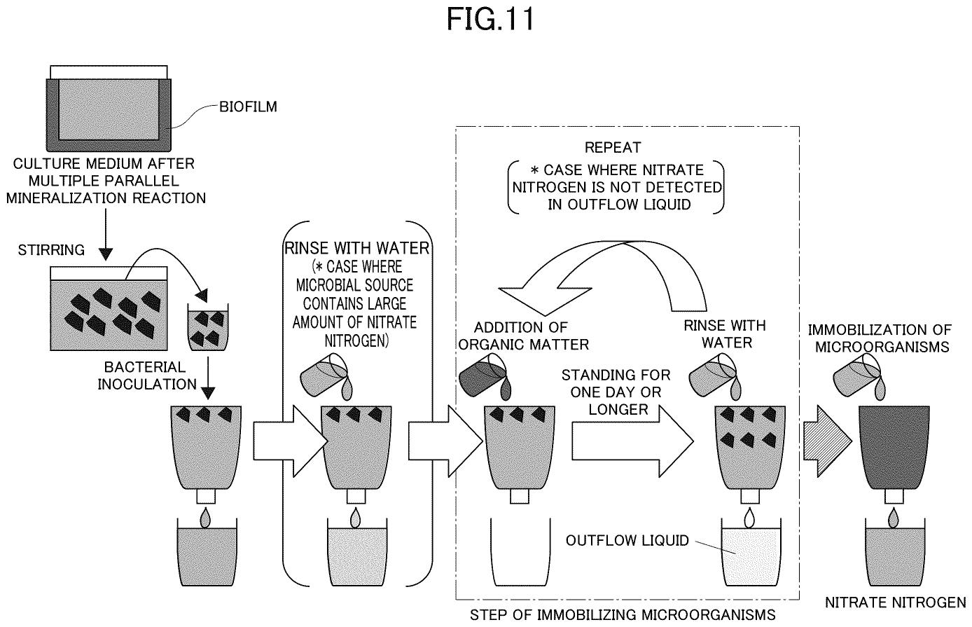

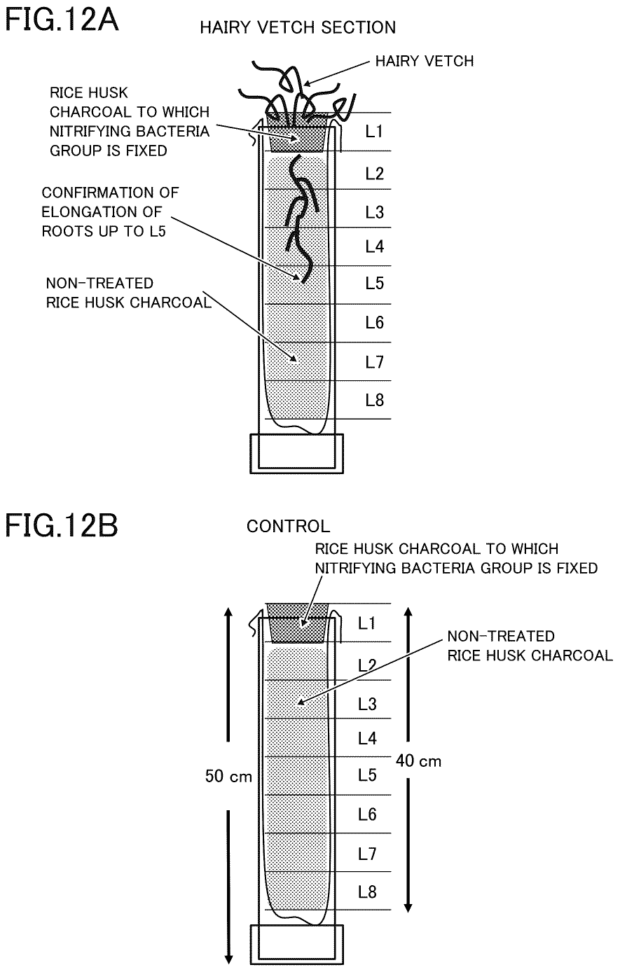

(1) Into a plastic pot having a diameter of 10 cm and having an opening at the bottom, 200 mL of sufficiently hydrated rice husk charcoal sterilized in an autoclave as a solid carrier was added, and a fixing treatment of a microbial population was performed on the rice husk charcoal according to the method of Patent Literature 1 and Non Patent Literature 1 (FIG. 11). That is, a bark compost (trade name: SANYO BARK, Sanyo Chip Industry Co., Ltd.) was added as a microbial source onto the rice husk charcoal, and addition of organic matter and rinsing with water were repeated to immobilize the microbial population to the rice husk charcoal.
(2) Sterile hairy vetch seeds were allowed to germinate for 3 days, and then planted in the pot in which a microbial population was immobilized in the (1) above. On the other hand, the control was one in which no hairy vetch was planted in the pot immobilized in the (1) above.
(3) Next, as a solid carrier, 2 L of rice husk charcoal that was sterilized in an autoclave and sufficiently hydrated was prepared and put in a vinyl bag. Then, the vinyl bag was filled in a vinyl chloride pipe having a length of 50 cm×a diameter of 9 cm.
(4) The pot in which the hairy vetch was planted as described in the (2) above was placed on an upper surface of the vinyl chloride pipe of the (3) above filled with untreated rice husk charcoal to which microorganisms were not immobilized (hairy vetch section; FIG. 12A). On the other hand, the control pot in which the hairy vetch was not planted as described in the (2) above was also placed on the upper surface of the vinyl chloride pipe filled with untreated rice husk charcoal in the same manner as described above (control; FIG. 12B). Each test section was tested in triplicate.
(5) On test day 0, 1.5 g of the skipjack broth was added to the upper portion of the pot of each test section, and thereafter, 0.1 g of the skipjack broth was added every 4 days. In addition, in order to prevent drying, 50 mL of milli-Q water was added to the upper portion of the pot of each test section every day. Cultivation was performed for 17 days under conditions of a day length of 16 h/day, a light intensity of 16,000 LUX, and a temperature of 24° C. The control was also treated in the same manner as above.
(6) After the cultivation, the rice husk charcoal filled in the hairy vetch section and the control was frozen at −20° C. The frozen rice husk charcoal was cut into eight layers L1 to L8 illustrated in FIG. 12, and 500 mg of the rice husk charcoal collected from each layer was pulverized in a microtube. Subsequently, 50 mg of the pulverized rice husk charcoal was collected, and DNA was extracted (about 100 ng/μL) using Nucleospin Soil (Takara Bio Inc.).
(7) From the DNA extracted in the (6) above, genes of the ammonia-oxidizing bacteria group, the nitrite-oxidizing bacteria group, and all bacteria were quantified using a real-time PCR method, and the amount of each bacteria in each layer was examined. Here, a primer targeting the amoA gene unique to the ammonia-oxidizing bacteria was used for the amplification of the ammonia-oxidizing bacteria group, a primer targeting the norB gene of the nitrite oxidoreductase was used for the amplification of the nitrite-oxidizing bacteria group (the genus Nitrobacter), and a primer targeting 16S rRNA was used for the amplification of all bacteria. Each primer is shown in Table 2.

TABLE 2

| | | SEQ ID | |
|---|---|---|---|
| Target | Primer/probe | NO: | References |
| Ammonia- | amoA1f (GGG GTT TCT ACT GGT GGT) | 1 | Rotthauwe et al. Appl. |
| oxidizing | amoA2r (CCC CTC KGS AAA GCC TTC | | Environ. Microbiol. |
| bacteria | TTC) | 2 | 63: 4704-4712. |
| amoA | | | |
| The genus | NxrB 1F (ACGTGGAGACCAAGCCGGG) | 3 | Vanparys et al. Syst. |
| Nitrobacter | NxrB 1R | | Appl. Microbiol. 30, |
| norB | (CCGTGCTGTTGAYCTCGTTGA) | 4 | 297-308. |
| All | All-F | 5 | Yoshida A, et al. J. |
| bacteria | (CGCTAGTAATCGTGGATCAGAATG) | | Clin. Microbiol. 41: |
| 16S rRNA | All-R (TGTGACGGGCGGTGTGTA) | | 863-866 |
| | | 6 | Suzuki et al. J. Clin. |
| | | | Microbiol. 42, 2255- |
| | | | 2257. |

(8) For real-time PCR, GVP-9600 (Shimadzu Corporation) was used, and TB Green (registered trademark) Premix Ex Taq™ II (Tli RNaseH Plus) and ROX plus (Takara Bio Inc.) were used to prepare PCR reaction solutions shown in Table 3.

TABLE 3

| | | | Final |
|---|---|---|---|
| | Used | | concen- |
| Reagent | amount | | tration |
| TB Green Premix Ex Taq II (2×) (Tli RNaseH Plus), | 10 | μL | 1× |
| ROX plus | | | |
| PCR Forward primer (10 μM) | 0.8 | μL | 0.4 μM |
| PCR Reverse primer (10 μM) | 0.8 | μL | 0.4 μM |
| Template DNA (<100 ng) | 2 | μL | |
| Sterile purified water | 6.4 | μL | |
| Total | 20 | μL | |

(9) PCR conditions were performed according to the shuttle PCR standard protocol shown in Table 4.

TABLE 4

Initial denaturation
95 °C 30 seconds
PCR reaction
Number of cycles: 50
95 °C 5 seconds
60 °C 30 seconds

(10) A Ct value was calculated by the 2nd Derivative Maximum method. In addition, a ratio of each nitrifying bacteria was calculated by comparing the Ct values. For example, an abundance ratio (2^{ΔCt}) of the ammonia-oxidizing bacteria to the total amount of bacteria was calculated by the following (Equation 1).

[Math. 1]

2^{ΔCt}=2^{(Ct 16s-Ct amoA)}×100% (Equation 1)

### Results

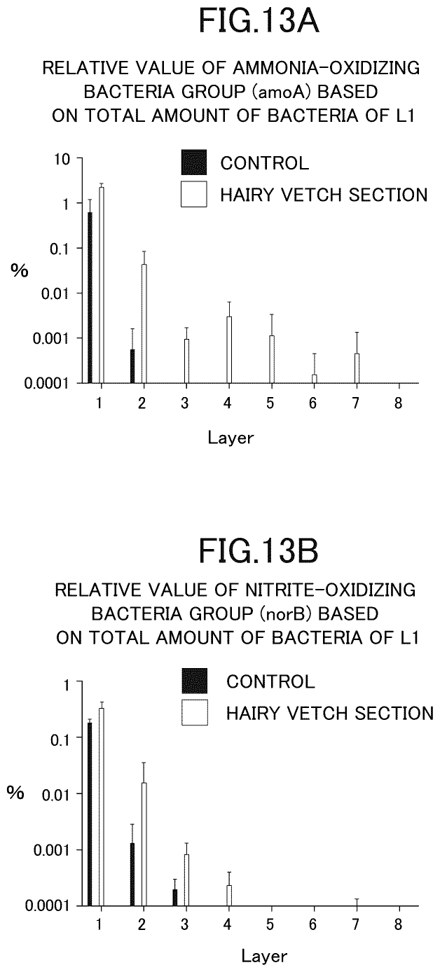

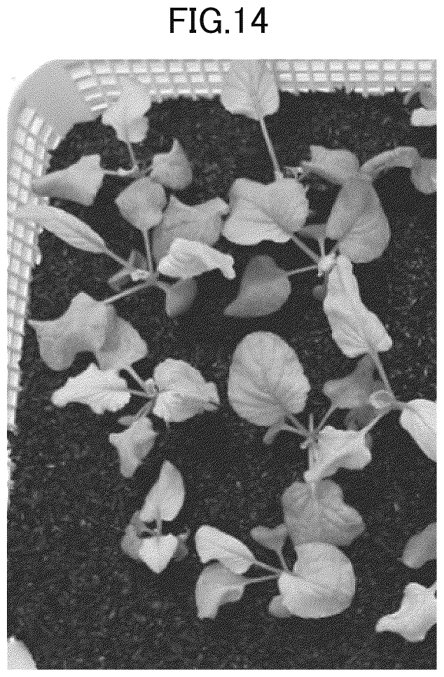

The results are illustrated in FIG. 13. FIG. 13 is a graph showing a relative value of the ammonia-oxidizing bacteria group (FIG. 13A) and a relative value of the nitrite-oxidizing bacteria group (FIG. 13B) with respect to the total amount of bacteria of L1 obtained by real-time PCR. The Y-axis indicates a relative value (%) on a logarithmic scale, and the X-axis indicates each layer of L1 to L8.

After 17 days of cultivation, as illustrated in FIG. 12A, the elongation of the roots up to L5 was observed in the hairy vetch section. As illustrated in FIG. 13A, in the control, the ammonia-oxidizing bacteria group was present only in L1 to which the nitrifying bacteria group was fixed in Test Method (1) and L2 immediately below L1, but in the hairy vetch section, the ammonia-oxidizing bacteria group was expanded together with the expansion of the rhizosphere, and the expansion up to L7 was confirmed. As illustrated in FIG. 13B, in the control, the nitrite-oxidizing bacteria group was present only in L1 to which the nitrifying bacteria group was fixed, L2 immediately below L1, and slightly L3, but in the hairy vetch section, it was confirmed that the nitrite-oxidizing bacteria were expanded and fixed up to L4 in which the expansion of the roots was sufficiently confirmed as compared with the control.

From these results, in the test section according to the present embodiment, it was possible to directly and quantitatively show that the nitrifying bacteria group expands inside the solid carrier as the rhizosphere of the plant expands, as compared with the control.

### COMPARATIVE EXAMPLES

A mesh basket was filled with 3 L of sufficiently hydrated rice husk charcoal, and 20 grains of Japanese mustard spinach seeds were sowed therein. For 18 days from 1 week after sowing, a liquid obtained by suspending 1 g of skipjack broth in 1 L of water was irrigated every day for cultivation. During the cultivation period, the cultivation was managed in a growth chamber at 25° C., a light period for 12 hours, and a dark period for 12 hours. After the end of cultivation, inorganic nitrogen contained in the rice husk charcoal was analyzed. The analysis was performed by RQ Flex (manufactured by Merck KGaA) using an ammonia test, a nitrite test, and a nitrate test of Reflect Quant (registered trademark).

The results are illustrated in FIG. 14. As illustrated in FIG. 14, in the present cultivation test, although the plants did not die, the leaves turned yellow and did not grow soundly. The analysis results of the inorganic nitrogen in the rice husk charcoal are shown in Table 5. From Table 5, only ammonium ions were detected, and nitrate ions and nitrite ions were not detected in the rice husk charcoal after the end of cultivation.

From this result, it became clear that unless a microbial source was added to the solid carrier, the soil formation of the solid carrier did not proceed, and the nitrification of organic matter did not proceed, such that the growth of plants was also deteriorated.

TABLE 5

| NH4⁺ | NO2⁻ | NO3⁻ |
|---|---|---|
| 12.1 | 0 | 0 |

The unit is mg/L.

Although the embodiments and Examples of the present disclosure have been described in detail with reference to the drawings, the specific configuration is not limited thereto, and a design change that does not depart from the gist of the present disclosure is included in the present disclosure.

For example, in the Examples, the method for producing a solid carrier having nitrate generating ability using rice husk charcoal as a solid carrier has been described, but the present disclosure is not limited thereto, and a case where any solid carrier having air permeability, for example, biochar, ceramics, rockwool, vermiculite, perlite, zeolite, sand, Kanuma soil, glass, urethane, nylon, a melamine resin, a wood chip, straw, sphagnum moss, cotton, paper, polyacrylamide gel, agar, pumice, a soil material, a soil material sintered body, field soil, or the like is used can also be included in the present disclosure.

In addition, for example, in the Examples, the method for producing a solid carrier having nitrate generating ability using skipjack broth as organic matter has been described, but the present disclosure is not limited thereto, and a case where any organic matter, for example, fish broth, fish meal, oil cake, garbage, a corn immersion liquid, rice bran, soybean flour, a plant body residue, an animal residue, milk, milk powder, livestock feces, human feces, insect feces, insect powder, algal powder, bacterial cells, a methane fermentation digestion liquid, sewage sludge, or the like is used can also be included in the present disclosure.

### INDUSTRIAL APPLICABILITY

The present disclosure is expected to be used by seedling production farms, seed potato production farms, and the like in which the occurrence of diseases is to be suppressed as much as possible, since non-soil media having no cultivation history can be formed into soil. In addition, the present disclosure is also applicable to a technique of cultivating plants in regolith (crushed material of minerals) of the Moon and the Mars or the like, and is expected to be industrially used.

What is claimed is:

1. A method for producing a solid carrier having nitrate generating ability, the method comprising a step of allowing all of the following (B) to (E) to coexist in the following solid carrier (A),
wherein a solid carrier to which the following microbial population (B) is immobilized is produced,
(A) a solid carrier having air permeability,
(B) a microbial population that generates nitrate nitrogen by mineralizing organic matter,
(C) organic matter,
(D) water, and
(E) a plant body that absorbs nitrate nitrogen.

2. The method according to claim 1, wherein the plant body (E) is allowed to coexist in the solid carrier (A) by sowing or planting a seed of the plant body (E) or a vegetatively propagatable tissue of the plant body (E) in the solid carrier (A).

3. The method according to claim 2, wherein after a step of adding and/or mixing the microbial population (B) to an upper layer portion of the solid carrier (A) is performed, a step of sowing or planting the seed of the plant body (E) or the vegetatively propagatable tissue of the plant body (E) in the solid carrier (A) is performed.

4. The method according to claim 2, wherein after a step of attaching the microbial population (B) to at least a part of a surface of the seed of the plant body (E) or the vegetatively propagatable tissue of the plant body (E) is performed, a step of sowing or planting the seed of the plant body (E) or the vegetatively propagatable tissue of the plant body (E) in the solid carrier (A) is performed.

5. The method according to claim 1, wherein the plant body (E) is allowed to coexist in the solid carrier (A) by planting the plant body (E) after germination in the solid carrier (A).

6. The method according to claim 5, wherein the method includes a step of growing the plant body (E) in soil containing the microbial population (B), and a step of planting the plant body (E) after germination obtained in the previous step in the solid carrier (A).

7. The method according to claim 5, wherein after a step of adding and/or mixing the microbial population (B) to an upper layer portion of the solid carrier (A) is performed, a step of planting the plant body (E) after germination in the solid carrier (A) is performed.

8. The method according to claim 1, wherein the solid carrier (A) is one or more selected from the group consisting of biochar, ceramics, rockwool, vermiculite, perlite, zeolite, sand, Kanuma soil, glass, urethane, nylon, a melamine resin, a wood chip, straw, sphagnum moss, cotton, paper, polyacrylamide gel, agar, pumice, a soil material, a soil material sintered body, and field soil.

9. The method according to claim 1, wherein the organic matter (C) is one or more selected from the group consisting of plant-derived, animal-derived, bacteria-derived, food-derived, and waste-derived organic matter.

10. The method according to claim 1, wherein the plant body (E) is one or more plant bodies selected from Asteraceae, Brassicaceae, Solanaceae, Amaranthaceae, Amaryllidaceae, Rosaceae, Fabaceae, Cucurbitaceae, Poaceae, Zingiberaceae, Liliaceae, Apiaceae, Chenopodiaceae, and Convolvulaceae.

11. The method according to claim 1, wherein the solid carrier (A) is used in a state of being filled in a container.

12. A plant cultivation method comprising producing a solid carrier having nitrate generating ability by the method according to claim 1, and cultivating a plant using the obtained solid carrier.

13. A soil composition for producing a solid carrier having nitrate generating ability, wherein all of the following (B) to (E) are allowed to coexist in the following solid carrier (A),
(A) a solid carrier having air permeability,
(B) a microbial population that generates nitrate nitrogen by mineralizing organic matter,
(C) organic matter,
(D) water, and
(E) a plant body that absorbs nitrate nitrogen.

14. The soil composition according to claim 13, wherein the solid carrier (A) has a nitrate recovery rate of 1% or more as measured by the following method,
nitrate recovery rate measuring method:
(1) the organic matter (C) is added to the solid carrier in an amount of nitrogen of 0.05 g or more per 1 L of the solid carrier and then allowed to stand at 24 to 26° C. for 1 hour or longer,
(2) the solid carrier is washed by adding 50 mL or more of water per 1 L of the solid carrier and allowing the water to flow out from the solid carrier,
(3) an outflow liquid flowed out from the solid carrier is recovered, and a content of nitrate nitrogen in the outflow liquid is measured, and
(4) a ratio of the content of nitrate nitrogen in the outflow liquid measured in the step (3) to the amount of nitrogen in the organic matter added in the step (1) is determined as a nitrate recovery rate.

15. The soil composition according to claim 13, wherein the solid carrier (A) is one or more selected from the group consisting of biochar, ceramics, rockwool, vermiculite, perlite, zeolite, sand, Kanuma soil, glass, urethane, nylon, a melamine resin, a wood chip, straw, sphagnum moss, cotton, paper, polyacrylamide gel, agar, pumice, a soil material, a soil material sintered body, and field soil.

16. The soil composition according to claim 13, wherein the organic matter (C) is one or more selected from the group consisting of plant-derived, animal-derived, microorganism-derived, food-derived, and waste-derived organic matter.

17. The soil composition according to claim 13, wherein the plant body (E) is one or more plant bodies selected from Asteraceae, Brassicaceae, Solanaceae, Amaranthaceae, Amaryllidaceae, Rosaceae, Fabaceae, Cucurbitaceae, Poaceae, Zingiberaceae, Liliaceae, Apiaceae, Chenopodiaceae, and Convolvulaceae.

18. The soil composition according to claim 13, wherein the solid carrier (A) is filled in a container.


SEQUENCE LISTING (6 sequences)

SEQ ID NO: 1 - DNA, 18 bp, synthetic construct
  source 1..18: mol_type = other DNA
ggggtttctactggtggt

SEQ ID NO: 2 - DNA, 21 bp, synthetic construct
  source 1..21: mol_type = other DNA
cccctckgsaaagccttcttc

SEQ ID NO: 3 - DNA, 19 bp, synthetic construct
  source 1..19: mol_type = other DNA
acgtggagaccaagccggg

SEQ ID NO: 4 - DNA, 21 bp, synthetic construct
  source 1..21: mol_type = other DNA
ccgtgctgttgayctcgttga

SEQ ID NO: 5 - DNA, 24 bp, synthetic construct
  source 1..24: mol_type = other DNA
cgctagtaatcgtggatcagaatg

SEQ ID NO: 6 - DNA, 18 bp, synthetic construct
  source 1..18: mol_type = other DNA
tgtgacgggcggtgtgta